(19) United States
(12) Patent Application Publication
(10) Pub. No.: US 2019/0099558 A1
(43) Pub. Date: Apr. 4, 2019

(54) Intravenous dripping system

(71) Applicant: Sang-Woon Lee, Seoul (KR)

(72) Inventor: Sang-Woon Lee, Seoul (KR)

(21) Appl. No.: 15/719,710

(22) Filed: Sep. 29, 2017

(51) Int. Cl. A61M 5/40; A61M 5/14; A61M 5/168; A61M 5/165; A61L 29/04

(52) U.S. Cl. CPC A61M 5/40; A61M 5/1413; A61L 29/041; A61M 5/16813; A61M 5/165; A61M 5/1411

(57) ABSTRACT

The present invention relates to a device for intravenous administration, and, more particularly, to an intravenous drip system capable of closing an outlet provided to a lower end of a drip vessel by lowering a floating valve in the drip vessel when a level of a medical solution filled in the drip vessel of the intravenous drip system falls down, thereby preventing air from flowing into a patent's vein.

### BACKGROUND OF THE INVENTION

### Field of the Invention

The present invention relates to a device for intravenous administration, and, more particularly, to an intravenous drip system capable of closing an outlet provided to a lower end of a drip vessel by lowering a floating valve in the drip vessel when a level of a medical solution filled in the drip vessel of the intravenous drip system falls down, thereby preventing air from flowing into a patent's vein.

### Background of the Related Art

In general, a device for intravenous administration is a medical instrument which is medically designed to introduce a medical solution into a patient's body. FIG. 1 shows a construction of a common intravenous drip system including a drip vessel 100 to receive the medical solution from a medical solution container 101, a drip tube 200 through which the medical solution flows, a needle 300 connected to an end of the drip tube 200, and a flow control valve 400 and a manifold 500 which are provided at an intermediate portion of the drip tube 200.

The drip vessel 100 is provided with a sharp tooth 110 at an upper end thereof, and the medicine container 101 is provided with a port 102 equipped with a rubber cap. The medical solution is withdrawn by inserting the sharp tooth 110 into the port 102. The drip vessel 100 is made of a soft transparent material, so that a healthcare worker can visually check discharge speed of the medical solution and can squeeze the drip vessel to accelerate the discharge of the medical solution. The medical solution withdrawn via the tooth 110 is temporarily stored in the drip vessel 100, and then is administered to a patient's body through the drip tube 200 and the needle 300.

The flow control valve 400 is designed to adjust a flow rate of the medical solution administered into the patient's body through the drip tube 200 or block a flow passage, and consists of a guide rail 410 and a pressing roller 420. If the medical solution stored in the container 101 runs out, the healthcare worker should block the flow control valve 400 in time so that air remained in the container 100 does not flow into a patient's vein.

In order to solve the above problem, some intravenous drip devices are disclosed in U.S. Pat. No. 4,959,053 (Sep. 25, 1990) and WO 2010/125479 (Nov. 4, 2010). FIG. 2 shows an example of the intravenous drip device disclosed in U.S. Pat. No. 4,959,053, in which a valve 4 is provided in a drip vessel 1. If a level of the medical solution rises in the drip vessel 1, the valve 4 goes up along the level of the medical solution due to buoyancy. However, if the level of the medical solution drops, the valve 4 goes down along the level of the medical solution to close an upper end of a drip tube 3. Therefore, it is possible to prevent an unexpected accident, i.e., air flows into the patient's vein through the drip tube 3, at night or under situation with no healthcare worker.

However, the above intravenous drip device has a problem in that filling the medical solution into the drip vessel and the drip tube 3 is very cumbersome work. In order to administer the medical solution to a patient by use of the intravenous drip device, a preparation process of extracting the air from the drip tube 3 and filling the drip vessel 1 and the drip tube 3 with the medical solution should be generally performed. In the case of the intravenous drip device according to the related art shown in FIG. 2, however, the flow control valve 4 closes an upper end of the drip tube 3 due to the weight thereof, before the drip vessel 3 is filled with the medical solution. Since the flow of the air is not free in the state in which the upper end of the drip tube 3 is closed by the flow control valve 4, it is very difficult to fill the drip vessel 1 and the drip tube 3 with the medical solution.

### SUMMARY OF THE INVENTION

Therefore, an object of the present invention is to provide an intravenous drip system capable of automatically blocking a flow path of a drip tube to prevent air from flowing to a patient's vein after the medical solution stored in a drip vessel runs out.

Another object of the present invention is to provide an intravenous drip system in which a preparation process of filling a drip vessel and a drip tube with a medical solution is very easy.

According to one aspect of the present invention, there is provided an intravenous drip system including: a port coupler configured to connect to a port provided to a medicine container to discharge a medical solution, and having a drip tube protruding from a center portion of a lower end thereof; a cylindrical drip vessel configured to engage to a lower flange of the port coupler, and having an inner space in which the medical solution dripped from the drip tube is temporarily stored, the cylindrical drip vessel being provided with an outlet at a lower end thereof; and a floating valve made of a material to float in the medical solution stored in the drip vessel, and having a floating cup suspending from the lower end of the drip tube, through-holes penetrating a lower end of the floating cup in a circumferential direction, and an air blocking valve engaged to the lower end of the floating cup.

The floating valve is detached from the drip tube by pressing both sides of the drip vessel or bending the drip vessel, and if a level of the medical solution rises in the drip vessel, the floating valve goes up to open the outlet, while if the level of the medical solution drops, the floating valve goes down so that the outlet is closed by the air blocking valve.

The intravenous drip system further includes a cylindrical elastic holder which is engaged to the lower end of the port coupler, and the floating cup is suspended from a lower end of the elastic holder.

With the above configuration of the intravenous drip system according to the present invention, after the medical solution stored in the drip vessel runs out when the medical solution is administered to a patient, the outlet of the drip vessel is automatically closed by the floating valve, thereby preventing air remaining in the drip vessel or drip tube from flowing to a patient's vein.

Also, since the floating valve is suspended from the lower end of the port coupler, and the floating valve is separated after the drip vessel is filled with the medical solution to some degree, a preparation process of filling the drip vessel and the drip tube with the medical solution is very easy.

### BRIEF DESCRIPTION OF THE DRAWINGS

FIG. 1 is a view illustrating a structure of an intravenous drip device according to the related art.

FIG. 2 is a view illustrating one example of a drip vessel designed to block air according to the related art.

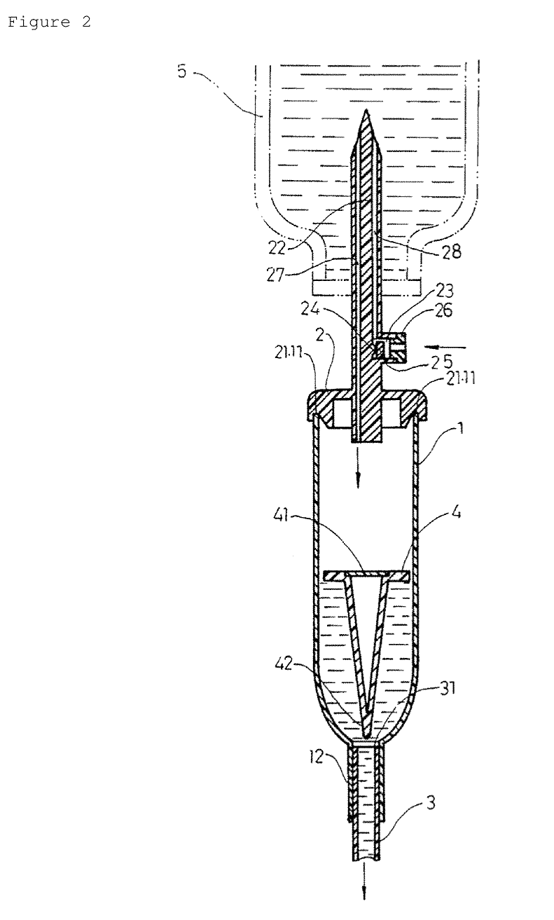

FIG. 3 is an exploded perspective view illustrating a drip vessel according to the first embodiment of the present invention.

FIG. 4 is a cross-sectional view illustrating an engagement state of an intravenous drip system according to the first embodiment.

FIG. 5 is an exploded perspective view illustrating a drip vessel according to the second embodiment of the present invention.

FIG. 6 is a cross-sectional view illustrating an engagement state of an intravenous drip system according to the second embodiment.

FIG. 7 is a cross-sectional view illustrating an operation state of the intravenous drip system according to the second embodiment.

### DETAILED DESCRIPTION OF THE PREFERRED EMBODIMENT

Hereinafter, embodiments of the present invention will be explained in detail in conjunction with the accompanying drawings so that those skilled in the art can easily carry out the present invention. In the following description, like reference numerals are attached to elements identical to those throughout the embodiment, and the description thereof is omitted herein.

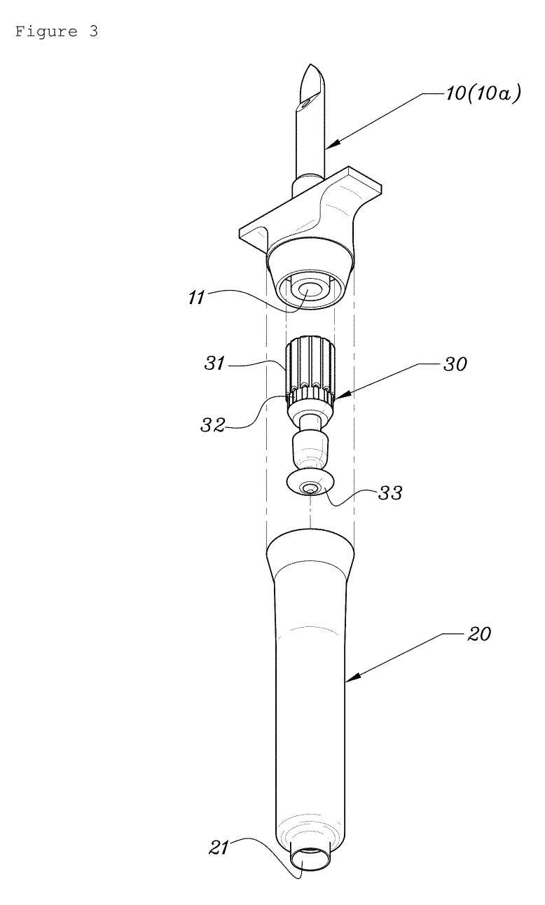

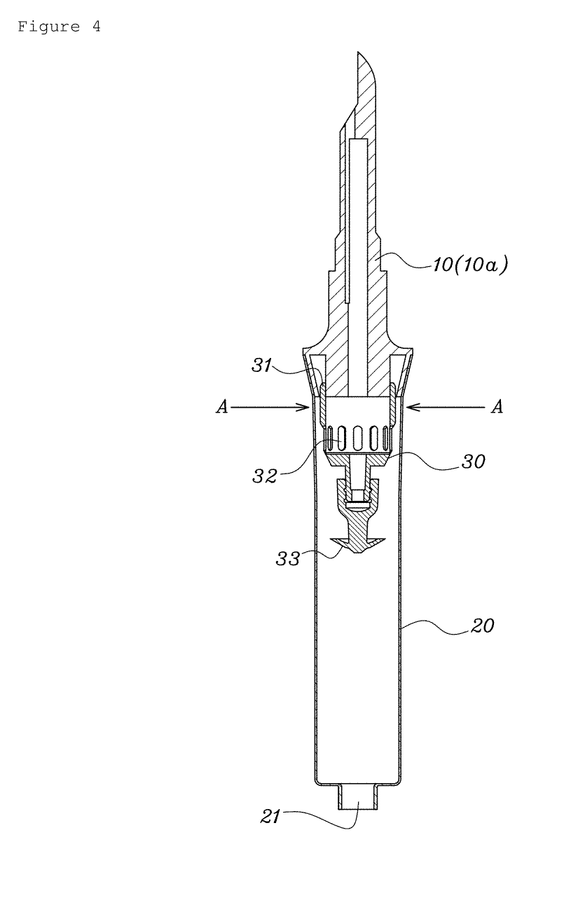

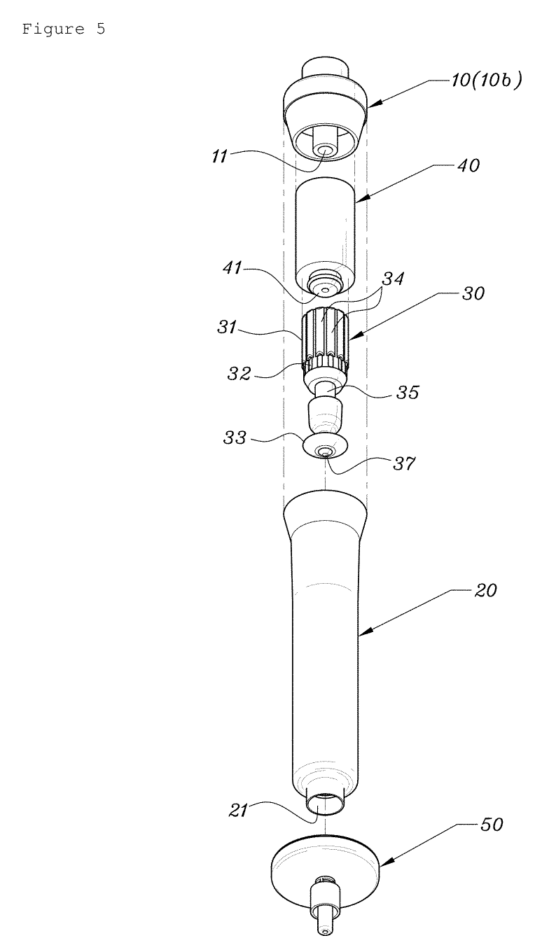

As illustrated in FIGS. 3 to 7, an intravenous drip system according to the present invention includes a port coupler 10, a cylindrical drip vessel 20, and a floating valve 30 provided in the drip vessel 20. FIGS. 3 and 4 show the first embodiment of the present invention in which a port coupler 10a of a sharp tooth type is provided, and FIGS. 5 to 7 show the second embodiment of the present invention in which a port coupler 10b of a luer-lock type is provided. A reference numeral 10 is added to components of the port couplers 10a and 10b which are widely known in the art.

First, the first embodiment of the present invention will be described in detail. As illustrated in FIGS. 3 and 4, the port coupler 10a according to the first embodiment is formed in the shape of a sharp tooth, and includes a drip tube 11 protruding from a center portion of a lower end thereof which is inserted in a port provided to a medicine container. A medical solution stored in the medicine container drops away from the medicine container through the drip tube 11.

An upper end of the cylindrical drip vessel 20 is engaged to a lower flange of the port coupler 10a, and has an inner space in which the medical solution dripped from the drip tube 11 is temporarily stored. The cylindrical drip vessel 20 is provided with an outlet 21 at a lower end thereof. The drip vessel 20 is substantially identical to a common drip vessel, and is preferably made of a transparent or translucent material so that a healthcare worker can observe an inner state. Also, the drip vessel 20 is preferably made of a soft material having an elastic restoring force, such as a polypropylene or polyethylene resin, so that a healthcare worker can squeeze the drip vessel to pump the medical solution, if necessary.

The floating valve 30 is provided in the drip vessel 20, and has a floating cup 31 suspending from the lower end of the drip tube 11. Through-holes 32 penetrate through a lower end of the floating cup 31 in a circumferential direction, and the medical solution 32 flows through the through-holes 32. An air blocking valve 33 is engaged to the lower end of the floating cup 31.

The floating cup 31 is made of a material which is harmless to a human body and has buoyance to float on the medical solution stored in the drip vessel 20. Preferably, the floating cup 31 is made of a polypropylene resin. For reference, the polypropylene resin has specific gravity of 0.91 lower than that of the medical solution thereby to float on the medical solution. Since the floating cup 31 is formed with the through-holes 32, the drip vessel 20 can be filled with the medical solution through the drip tube 11 and the floating cup 31 even in the state in which the floating cup 31 is suspended from the lower end of the drip tube 11.

Preferably, the air blocking valve 33 is formed in the shape of a disc so as to be effectively brought into contact with a periphery of the outlet 21, and is configured in such a way that a thickness is gradually decreased from a center to an edge. The air blocking valve 33 is preferably made of a soft material, such as elastomer, silicone or rubber.

The upper end of the floating valve 30 is suspended from the lower end of the drip tube 11, as illustrated in FIG. 4. The term “suspended” herein means the state in which the floating valve is temporarily engaged to the drip tube, but is separated from the drip tube if an appropriate external force is applied thereto. If both sides of the drip vessel 20 are pressed or are slightly bent in the direction of the arrow A in FIG. 4, the floating cup 31 is slidably moved away from the lower end of the drip tube 11, so that the floating valve 30 is detached from the drip tube 11.

According to the intravenous drip system according to the first embodiment, as illustrated in FIG. 4, the drip vessel 20 and the drip tube (not shown in FIG. 4) connected to the lower end of the drip vessel 20 are filled with the medical solution, with the air being removed therefrom, in the state in which the floating valve 30 is suspended from the lower end of the drip tube 11. In this instance, the medical solution flowing to the drip tube 11 flows out from the floating cup 31 through the through-holes 32, and then is collected in the lower end of the drip vessel 20. If the drip vessel 20 and the drip tube are filled with the medical solution, the floating valve 30 is detached from the drip tube 11.

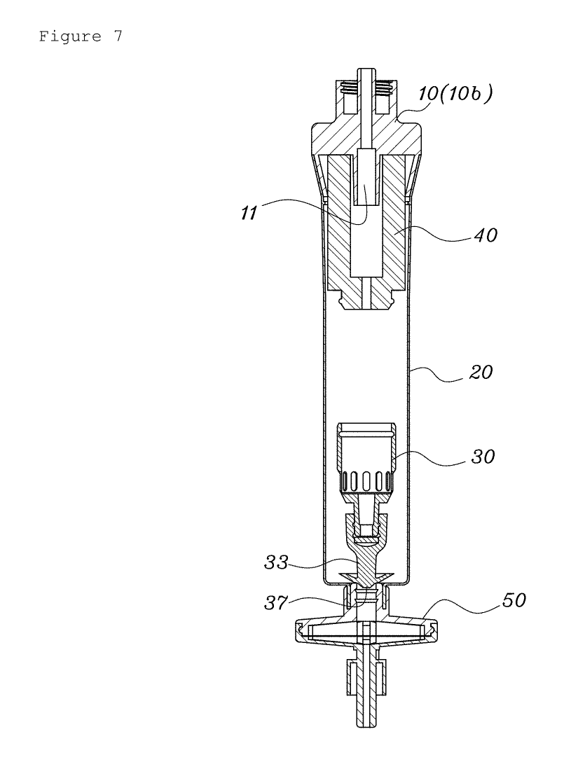

Now, the second embodiment of the present invention will be described in detail. As illustrated in FIGS. 5 and 7, the port coupler 10b according to the second embodiment is formed in the type of a luer-lock which is screwed to a port of the medical container, with no the sharp tooth, and includes a drip tube 11 protruding from a center portion of a lower end thereof. A cylindrical elastic holder 40 is engaged to a lower end of the port coupler 10b, and the floating cup 31 of the floating valve 30 is suspended from the lower end of the elastic holder 40.

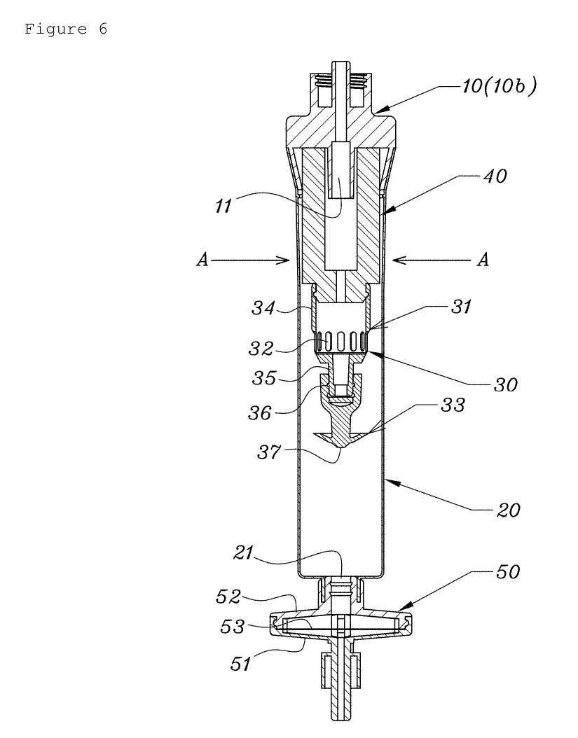

The elastic holder 40 is designed to easily detach the floating valve 30 from the drip tube. The elastic holder 40 is preferably made of elastomer, silicon or a rubber material. If both sides of the drip vessel 20 are pressed or are slightly bent in the direction of the arrow A in FIG. 6, the floating valve 30 suspended from the elastic holder 40 is detached from the drip tube by the pressure.

An outer peripheral portion of the elastic holder 40 is brought into close contact with an inner peripheral portion of the drip vessel 20 so that the pressure generated when the drip vessel 20 is pressed can be easily transferred to the elastic holder 40. A coupling protrusion 41 protrudes from the lower end of the elastic holder 40, and is temporarily engaged to the upper end of the floating cup 31. The medical solution dripping from the drip tube 11 flows down in the drip vessel 20 through the elastic holder 40 and the coupling protrusion 41.

Although only the case where the elastic holder 40 is engaged to the port coupler 10b is illustrated in the second embodiment of the present invention, the elastic holder 40 may be provided to the port coupler 10a of the first embodiment.

As illustrated in FIGS. 5 and 6, the outer peripheral surface of the floating cup 31 may be provided with a spline 34 in a vertical direction. The spline 34 is designed to take the floating cup apart from the inner surface of the drip vessel 20 by a given interval, thereby enabling the floating cup to move in a vertical direction and the medical solution to pass. Three or eight splines 34 are preferably provided in a circumferential direction of the floating cup 31.

A coupling protrusion 35 protrudes from the lower end of the floating cup 31, and is engaged to the air blocking valve 33. The top surface of the air blocking valve 33 is provided with an engaging groove 36 to which the coupling protrusion 35 of the floating cup 31 is engaged. The floating cup 31 and the air blocking valve 33 are integrally engaged to each other by forcible interference between the coupling protrusion 35 and the engaging groove 36.

A sealing stopper 37 protrudes from the bottom surface of the air blocking valve 33, and is inserted in the outlet 21. The sealing stopper 37 is designed to place the floating valve 30 in place, and thus when the floating valve 30 moves down, the floating valve is not slanted to completely block the outlet 21.

The intravenous drip system according to the present invention may include a filtering unit 50 to filter the medical solution discharged from the outlet 21. The filter unit 50 has a first filter housing 51, a second filter housing 52, and a membrane 53, as illustrated in FIG. 6. The first filtering housing 51 and the second filtering housing 52 are formed in the shape of a disc, and are placed opposite to each other. The membrane 53 is provided in an inner space formed between the first and second filtering housings.

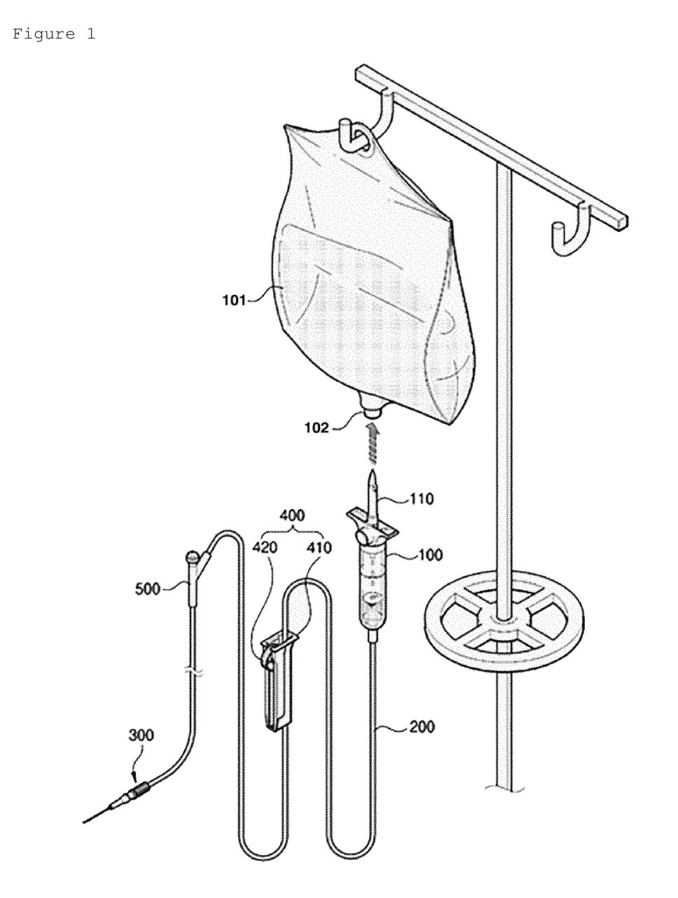

Supposing the intravenous drip system according to the second embodiment is installed to the common intravenous drip device in FIG. 1, the method of using the intravenous drip system according to the present invention will now be described. First, in the state in which the upper end of the floating cup 31 is inserted into the lower end of the elastic holder 40 and then is suspended therefrom, the flow control valve 400 provided in the drip tube 200 is closed, and the drip vessel 20 is filled with the medical solution by squeezing the lower end of the drip vessel 20. After that, the flow control valve 400 is opened to remove the air from the drip tube 200.

It should be noted that the floating valve 30 may be detached from the elastic holder 40 if the portion around the elastic holder 40 is pressed when the drip vessel 20 is squeezed. The floating valve 30 is detached from the drip tube 11 by pressing the upper portion of the drip vessel 20, that is, the intermediate portion (indicated by the arrow A) of the elastic holder 40, in the state in which the drip vessel 20 is filled with the medical solution by almost half of the drip vessel. Then, the medical solution is administered into a patient's body, while the flow rate of the medical solution is adjusting by the flow control valve 400.

The floating valve 30 detached from the drip tube 11 floats vertically along the level of the medical solution in the drip vessel 20. In the state in which the drip vessel 20 is filled with the medical solution, the floating valve 30 floats in the medical solution to open the outlet 21. If the level of the medical solution drops, the floating valve 30 goes down, so that the outlet 21 is closed by the air blocking valve 33.

FIG. 6 shows the state in which the medical solution is completely discharged from the drip vessel 20, and the floating valve 30 goes down along the level of the medical solution, so that the upper end of the outlet 21 is closed by the air blocking valve 33.

When the floating valve 30 goes down, the floating cup settles the position of the floating valve 30 so that the floating valve 30 moves down in a straight way in the state in which the floating valve is not slanted. If the floating valve 30 is slanted, the air blocking valve 32 is not correctly seated on the outlet 21. The sealing stopper 37 enables the air blocking valve 33 to completely close the outlet 21 when the floating valve 30 goes down.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

What is claimed is:

1. An intravenous drip system comprising:
a port coupler configured to connect to a port provided to a medicine container to discharge a medical solution, and having a drip tube protruding from a center portion of a lower end thereof;
a cylindrical drip vessel configured to engage to a lower flange of the port coupler, and having an inner space in which the medical solution dripped from the drip tube is temporarily stored, the cylindrical drip vessel being provided with an outlet at a lower end thereof; and
a floating valve made of a material to float in the medical solution stored in the drip vessel, and having a floating cup suspending from the lower end of the drip tube, through-holes penetrating a lower end of the floating cup in a circumferential direction, and an air blocking valve engaged to the lower end of the floating cup,
wherein the floating valve is detached from the drip tube by pressing both sides of the drip vessel, and if a level of the medical solution rises in the drip vessel, the floating valve goes up to open the outlet, while if the level of the medical solution drops, the floating valve goes down so that the outlet is closed by the air blocking valve.

2. The intravenous drip system according to claim 1, wherein a cylindrical elastic holder is engaged to the lower end of the port coupler, and the floating cup is suspended from a lower end of the elastic holder.

3. The intravenous drip system according to claim 1, wherein the floating cup of the floating valve is made of a polypropylene resin, and the air blocking valve is made of a soft elastomer, silicone or rubber material.

4. The intravenous drip system according to claim 1, wherein an outer peripheral surface of the floating cup is provided with a spline in a longitudinal direction, a coupling protrusion protrudes from the lower end of the floating cup, a top surface of the air blocking valve is provided with an engaging groove to which the coupling protrusion is engaged, and a sealing stopper protrudes from a bottom surface of the air blocking valve and is inserted into the outlet.

5. The intravenous drip system according to claim 1, wherein the lower end of the drip vessel is provided with a filtering unit to filter the medical solution discharged from the outlet.

6. The intravenous drip system according to claim 2, wherein an outer peripheral portion of the elastic holder is brought into close contact with an inner peripheral portion of the drip vessel, and a coupling protrusion protrudes from the lower end of the elastic holder, and is temporarily engaged to an upper end of the floating cup.

7. The intravenous drip system according to claim 2, wherein the floating cup of the floating valve is made of a polypropylene resin, and the air blocking valve is made of a soft elastomer, silicone or rubber material.

8. The intravenous drip system according to claim 2, wherein an outer peripheral surface of the floating cup is provided with a spline in a longitudinal direction, a coupling protrusion protrudes from the lower end of the floating cup, a top surface of the air blocking valve is provided with an engaging groove to which the coupling protrusion is engaged, and a sealing stopper protrudes from a bottom surface of the air blocking valve and is inserted into the outlet.

9. The intravenous drip system according to claim 2, wherein the lower end of the drip vessel is provided with a filtering unit to filter the medical solution discharged from the outlet.
